(19) United States
(12) Patent Application Publication
(10) Pub. No.: US 2021/0177634 A1
(43) Pub. Date: Jun. 17, 2021

(54) Fastening strip for knee bandage

(71) Applicant: Jingrong Lan, Wuping County (CN)

(72) Inventor: Jingrong Lan, Wuping County (CN)

(21) Appl. No.: 17/184,623

(22) Filed: Feb. 25, 2021

(51) Int. Cl. A61F 5/01

(52) U.S. Cl. CPC A61F 5/0109

(57) ABSTRACT

A fastening strip for knee bandage includes a holding part at a first end; an insertion part at a second end; and a flexible tape section having a first end formed with the holding part and a second end formed with the insertion part. The flexible tape section has two symmetric, wavy sides. The holding part is cross-shaped. The insertion part is flat and shaped as a triangle.

### BACKGROUND OF THE INVENTION

### 1. Field of the Invention

The invention relates to fastening strips for knee bandage and more particularly to such a fastening strip for knee bandage and a knee bandage both having improved characteristics.

### 2. Description of Related Art

A bandage is a piece of material used either to support a medical device such as a dressing or splint, or on its own to provide support to or to restrict the movement of a part of the body.

However, conventional fastening strips for knee bandage are disadvantageous because they are uneasy to put on the knee and their holding capability is low. Thus, the need for improvement still exists.

### SUMMARY OF THE INVENTION

The invention has been made in an effort to solve the problems of the conventional art including being uneasy to put on the knee and low holding capability by providing a fastening strip for knee bandage having novel and nonobvious characteristics.

To achieve above and other objects of the invention, the invention provides a fastening strip for knee bandage comprising a holding part at a first end; an insertion part at a second end; and a flexible tape section interconnecting the holding part and the insertion part; wherein the flexible tape section includes a first connection member formed with the holding part, a second connection member formed with the insertion part, and an elongated interconnection having a first side and a wavy second side; wherein the holding part is cross-shaped; and wherein the insertion part is flat and shaped as a triangle.

Preferably, the first connection member has two symmetric wavy surfaces on two sides respectively; the second connection member has two symmetric wavy surfaces on two sides respectively; and the first side of the flexible tape section is smooth.

The fastening strip for knee bandage of the invention has the following advantageous effects in comparison with the prior art: the fastening strip can take hold the knee. The cross-shaped holding part increases holding capability. The insertion part is flat and shaped as a triangle for facilitating insertion into a knee bandage. The wavy sides provide increased flexibility and can withstand compression and bending.

The above and other objects, features and advantages of the invention will become apparent from the following detailed description taken with the accompanying drawings.

### BRIEF DESCRIPTION OF THE DRAWINGS

FIG. 1 is a perspective view of a fastening strip for knee bandage according to the invention; and

FIG. 2 is a perspective view of a knee bandage according to the invention having two fastening strips mounted thereon.

### DETAILED DESCRIPTION OF THE INVENTION

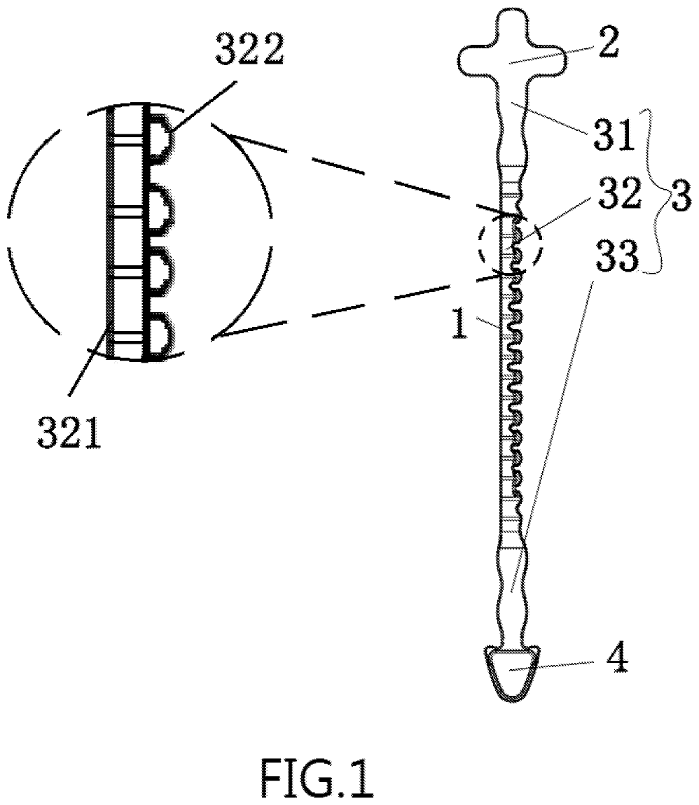

Referring to FIG. 1, a fastening strip 1 for knee bandage in accordance with the invention is made of flexible material (e.g., plastic) and comprises a holding part 2 at a first end, an insertion part 4 at a second end, and a flexible tape section 3 interconnecting the holding part 2 and the insertion part 4 as discussed in detail below.

The flexible tape section 3 includes a first connection member 31 formed with the holding part 2 and having two symmetric wavy surfaces on two sides respectively, a second connection member 33 formed with the insertion part 4 and having two symmetric wavy surfaces on two sides respectively, and an elongated interconnection 32 having a smooth first side 321 and a wavy second side 322 having equal segments. Alternatively, the second side 322 is toothed, has a plurality of half circular portions, or has a plurality of oval portions in other embodiments.

The holding part 2 is cross-shaped for increasing its holding capability. The insertion part 4 is flat and shaped as a triangle for facilitating insertion into a knee bandage.

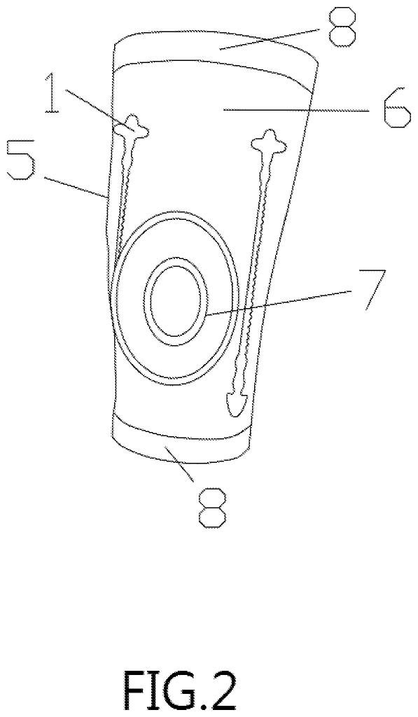

Referring to FIG. 2, a knee bandage 5 in accordance with the invention is shown and comprises a fabric part 6, and two constriction parts 8 at first and second ends of the fabric part 6 respectively for increasing safety when putting on the knee. Tension of the fabric part 6 is greater than that of the constriction parts 8 so that they are adapted to resiliently put on the knee. A pad 7 is inserted between the fabric part 6 and the knee and secured to the fabric part 6 using adhesive. Further, the knee is covered by the pad 7.

Two fastening strips 1 are provided at the two sides of the knee bandage 5. The fastening strips 1 are disposed lengthwise along the knee bandage 5 in use. The fastening strips 1 are secured to the knee bandage 5 using adhesive. Alternatively, only one fastening strip 1 is mounted on the knee bandage 5 in other embodiments.

The invention has the following advantageous effects in comparison with the prior art: the holding part 2 is adapted to taking hold of the knee bandage 5 and in turn the knee bandage 5 may hold the knee tightly.

While the invention has been described in terms of preferred embodiments, those skilled in the art will recognize that the invention can be practiced with modifications within the spirit and scope of the appended claims.

What is claimed is:

1. A fastening strip for knee bandage, comprising:
a holding part at a first end;
an insertion part at a second end; and
a flexible tape section having a first end formed with the holding part and a second end formed with the insertion part;
wherein the flexible tape section has two symmetric, wavy sides;
wherein the holding part is cross-shaped; and
wherein the insertion part is flat and shaped as a triangle.

2. The fastening strip for knee bandage of claim 1, wherein the flexible tape section includes a first connection member formed with the holding part, a second end formed with the insertion part, and an elongated interconnection formed with the first and second connection members.

3. The fastening strip for knee bandage of claim 2, wherein the first connection member has two symmetric wavy surfaces on two sides respectively; and wherein the second connection member has two symmetric wavy surfaces on two sides respectively.

4. The fastening strip for knee bandage of claim 2, wherein the elongated interconnection has a wavy first side.

5. The fastening strip for knee bandage of claim 4, wherein the wavy first side has a plurality of equal segments.

6. The fastening strip for knee bandage of claim 1, wherein the second side is toothed, has a plurality of half circular portions, or has a plurality of oval portions.

7. The fastening strip for knee bandage of claim 1, wherein the fastening strip is made of flexible material.

8. A knee bandage made of flexible material, comprising a pad corresponding to the knee and at least one fastening strip mounted on at least one side of the pad.

9. The knee bandage of claim 8, further comprising two constriction parts at first and second ends of the knee bandage respectively.
